(19) United States
(12) Patent Application Publication
(10) Pub. No.: US 2009/0111140 A1
(43) Pub. Date: Apr. 30, 2009

(54) SYSTEMS AND METHODS FOR CELL MEASUREMENT UTILIZING ULTRASHORT T2*

(75) Inventors: Wei Liu, Rockville, MD (US); Hannes Dahnke, Hamburg (DE); Tobias Schaeffter, Blackheath (GB)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/295,386

(22) PCT Filed: Mar. 22, 2007
(86) PCT No.: PCT/IB07/51013

§ 371 Date: Sep. 30, 2008

Related U.S. Application Data
(60) Provisional application No. 60/788,473, filed on Mar. 31, 2006.

(51) Int. Cl. C12Q 1/02; C12M 1/34

(52) U.S. Cl. 435/29; 435/287.1

(57) ABSTRACT

The present disclosure is directed to a new technique for MR measurement of ultrashort T₂* relaxation utilizing spin-echo acquisition. The ultrashort T₂* relaxometry can be used for the quantification of highly concentrated iron labeled cells in cell trafficking and therapy. In an exemplary embodiment, a signal is induced by a low flip angle RF pulse. Following excitation pulse, a gradient readout is applied to form an echo. The time between the RF pulse and the center of the gradient readout is defined as TE. In tissues with highly concentrated iron labeled cells, T₂* could be below 1 millisecond. Therefore, the signal can be decayed to a noise level with an echo time of a couple milliseconds. Because T₂ is much longer in SPIO labeled cells, the signal acquired by spin echo is much bigger than that from the gradient echo, thus avoiding the negative effects associated with the massive signal loss in the image. The ultrashort T₂* relaxation map can then by overlaid on the regular T₂* map to generate the final T₂* map of the field of view.

### BACKGROUND

1. Technical Field

The present disclosure relates to systems and methods that measure fast decaying T₂* relaxation for effective quantification of labeled cells using magnetic resonance imaging. The disclosed systems and methods are useful in a variety of applications, including cell trafficking and cell therapy.

2. Background Art

Cellular therapies using stem cells and immune cells for the purpose of repair and revascularization are being increasingly applied in clinical trials. Accurate delivery of cells to target organs can make the difference between failure and success. Quantifying the number of cells delivered in target tissue(s) is of great importance to optimize dose and timing of cellular therapy. Superparamagnetic iron oxide (SPIO) agents have been used to label cells ex vivo, providing researchers with the ability to monitor the migration, proliferation and homing of these cells with magnetic resonance (MR) imaging. SPIO labeling causes a strong relaxation rate (R₂) effect that increases linearly with iron concentration. R₂* is defined as 1/T₂*.

T₂* relaxometry is usually achieved by multiple gradient echo imaging. In tissues containing highly concentrated iron labeled cells, T₂* can be ultrashort. In exemplary instances, T₂* is below 1 to 2 milliseconds, although precise T₂* periods vary from application-to-application. The echo time of gradient echo is generally limited by hardware settings. It is not trivial to achieve ultrashort echo time in practical settings. Therefore, the signal decay in tissues with ultrashort T₂* is generally too rapid for regular gradient echo imaging.

Despite efforts to date, a need remains for systems and/or methods that overcome difficulties and limitations associated with conventional cell quantification techniques. In addition, a need remains for cell quantification techniques that do not require hardware modification(s) and/or dedicated RF pulse designs. Still further, systems and methods are needed for effectively and reliably monitoring and/or quantifying labeled cell levels, e.g., labeled stem cells, in various applications, including cellular therapies and the like. These and other needs are satisfied by the systems and methods disclosed.

### SUMMARY

The present disclosure provides systems and methods for measuring and/or quantifying cell levels in various applications, e.g., cell trafficking and cell therapy. Exemplary embodiments of the disclosed systems and methods involve the use of cells that have been labeled ex vivo with a contrasting agent or other identifying characteristic. The labeled cells are monitored using MR imaging so as to assess the migration, proliferation and/or homing of the labeled cells. Typically, the contrasting agent is SPIO, although alternative contrasting agents may be employed without departing from the spirit or scope of the present disclosure.

According to the present disclosure, T₂* relaxometry is advantageously employed in measuring labeled cell concentrations in a variety of cell-related applications. Since T₂* is ultrashort in highly concentrated iron labeled cells, advantageous systems and methods for measuring T₂* relaxometry are disclosed herein, such systems and methods using a sequence of spin echo imaging rather than the standard gradient echo imaging to achieve desirable results. In exemplary instances, T₂ is below 1 to 2 milliseconds, although the disclosed systems and methods have advantageous application across a broad range of T₂* values, such T₂* values generally varying from application-to-application. The disclosed systems and methods induce a regular spin echo signal generating a first spin echo image, followed by inducing multiple spin echo signals generating a series of additional spin echo images from suitable echo shifts towards said T₂* decay, and then deriving T₂*maps using exponential fitting.

Spin echo signals exiting the cells for MR imaging are formed by a first radio frequency (RF) pulse followed by a second RF pulse, respectively. Using spin echo signals, a T₂ curve is generated wherein T₂ is much longer for cells labeled with SPIO particles/nanoparticles than T₂* and defined by Mₛₛe^{-t/T}. The T2* decay curve of the spin echo is then defined by Mₛₛe^{TE/T2}e^{-(t-TE)/T2}*. The multiple spin echo images are taken at different intervals defined by an echo shift step that could be less than 1 ms. An ultrashort T₂* map is generated by the first spin echo image and the multiple spin echo images with suitable echo shifts by exponential fitting. An overall T₂* map is generated by overlying the ultrashort T₂* map on a regular T₂ map.

Additional features, functions and benefits of the disclosed systems and methods will be apparent from the description which follows, particularly when read in conjunction with the appended figures.

### BRIEF DESCRIPTION OF THE DRAWINGS

To assist those of ordinary skill in the art in making and using the disclosed systems and methods, reference is made to the appended figures, wherein:

FIG. 1 is a schematic for a standard T₂* relaxometry using multiple gradient echo sequence;

FIG. 2 is a schematic for an ultrashort T₂* relaxometry sequence using spin echo sequence;

FIG. 3a is an axial gradient echo image of a tumor rat;

FIG. 3b is an axial spin echo image with an echo shift of 0.8 ms;

FIG. 3c is a plussian blue strained tumor slice;

FIG. 4a is a regular T₂* map masked by a signal threshold to remove noise;

FIG. 4b is an ultrashort T₂ map overlaid on a regular T₂ map;

FIG. 5a is representative R₂* maps of labeled flank tumors;

FIG. 5b is representative R₂* maps of unlabeled flank tumors;

FIGS. 6(a)-6(c) are histograms of tumors with different number of iron labeled cells; and

FIG. 7 is a graph illustrating the linear correlation of R₂* with the number of labeled cells/mm³.

### DESCRIPTION OF EXEMPLARY EMBODIMENT(S)

Systems and methods are disclosed for measuring and/or quantifying cell levels, without the need for hardware modifications and/or dedicated RF pulse designs. The disclosed systems/methods have wide ranging utility, including cell trafficking and cell therapy applications. Labeled cells are monitored using MR imaging so as to assess the migration, proliferation and/or homing thereof. Fast decaying T₂* relaxation times are measured using MR imaging so as to effectively quantify the labeled cells, as described herein.

SPIO agents influence the T₁, T₂ and T₂* relaxation time. For cellular compartmental SPIO, the effect on T₂* relaxation is ten times higher than on T₂ relaxation. As a result, T₂ is much longer than T₂* in SPIO-labeled cells. The disclosed systems and methods utilize the relatively long T₂ decay by acquiring a series of spin echo images to advantageously facilitate a determination of the T₂* value, despite the massive signal loss associated with the ultrashort T₂* decay.

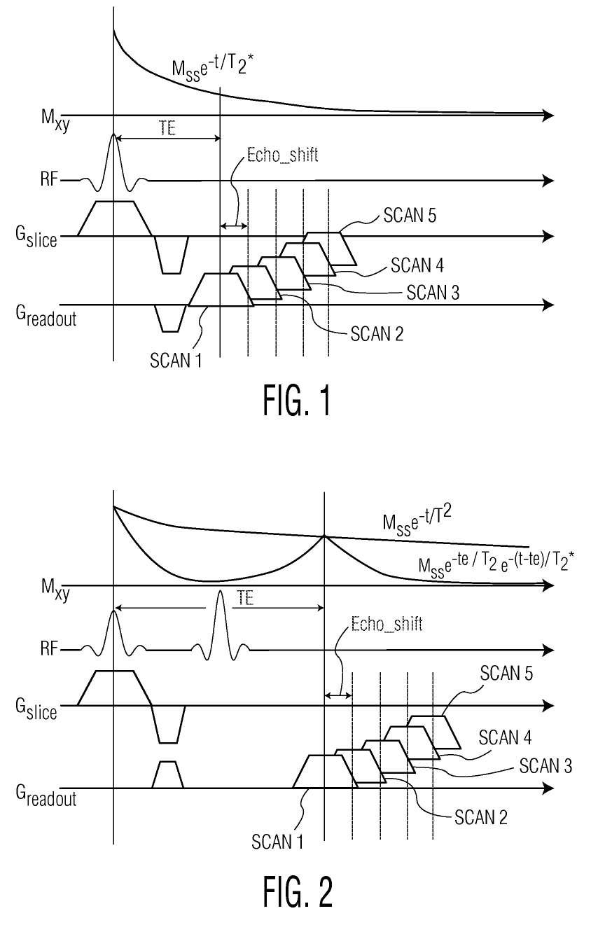

FIG. 1 illustrates a basic schematic of regular T₂* relaxometry using multiple gradient echo sequence. The signal is induced by a low flip angle RF pulse. Following an excitation pulse, a gradient readout is applied to form an echo. The time between the RF pulse and the center of the gradient readout is defined as “TE”. It is apparent that the time interval TE is restricted by the RF pulse and gradient waveform of the slice selection gradient and readout gradient. Thus, TE is limited by hardware settings, including specifically gradient strength and gradient rising time.

The signal acquired at the gradient echo is defined by Mₛₛe^{-TE/T2}*, where Mₛₛ is the magnetization at steady state. In tissues with highly concentrated iron labeled cells, T₂* could be below 1 or 2 milliseconds. Therefore, the signal can decay to a noise level with an echo time of a couple milliseconds. Prior efforts to reduce the TE have involved the modification of the hardware or large amount of work on the sequence design, neither approach being optimal and/or practical for many conventional applications.

FIG. 2 schematically illustrates various parameters associated with an exemplary implementation of the present disclosure. A spin echo is used to acquire an image according to the disclosed systems and methods. The use of spin echo substitutes for the conventional use of gradient echo. In an exemplary embodiment of the present disclosure, the spin echo is formed by a 90 degree RF pulse, followed by a 180 RF pulse. The signal intensity at TE is determined by the relationship: Mₛₛe^{-TE/T2}. Since T₂ is much longer in SPIO-labeled cells, the signal acquired by spin echo is much bigger than that from gradient echo, thus avoiding the negative effects associated with massive signal loss in the image. The ultrashort T₂* relaxation map can then by overlaid on a regular T₂* map to generate a final T₂* map for the field of view.

Measurement of ultrashort T₂* relaxation can be achieved by acquiring a series of spin echo images as shown in FIG. 2. The first echo is obtained as a regular spin echo image. The next images are acquired by shifting the readout towards the T₂* decay curve by suitable echo shift steps that could be below 1 millisecond. This method allows sampling of the T₂* decay curve created by the spin-echo signal. T₂* maps can then be derived using exponential fitting.

With further reference to FIG. 2, a series of images are acquired with spin echo sequence. The first scan is acquired as the standard spin echo image. The following scans (scan 2-scan 5) are acquired with echo shift towards the T₂* decay curve defined by the relationship: Mₛₛe^{-TE/T2}e^{-(t-TE)/T2}*. As demonstrated in FIG. 2, the disclosed systems and methods are effective in overcoming the limitations associated with the rapid decay associated with T₂* through advantageous spin echo utilization.

To further illustrate the uses and advantages associate with the disclosed systems and methods, reference is made to the following examples. However, it is to be understood that such examples are not limiting with respect to the scope of the present disclosure, but are merely illustrative of exemplary implementations and/or utilities thereof:

### Example 1

To facilitate measurement of fast decaying T₂* relaxation in tissues containing highly concentrated iron labeled cells, where T₂* decay is too rapid for regular multiple gradient echo T₂* mapping, the following methodology was employed. In vivo MR experiments in rats with iron labeled tumors were used to demonstrate that the disclosed methodology can be used to quantify ultrashort T₂* down to 1 to 2 milliseconds or less. Combined with regular T₂* mapping, the disclosed technique may be used to improve in vivo quantification and monitoring of tissues containing heavily iron labeled cells.

SPIO nanoparticles are widely used to influence the T₁, T₂ and T₂* relaxation times of labeled cells and tissues. The T₂* relaxation time is the most sensitive parameter to detect SPIO-labeled cells and, based on the advantageous systems and methods disclosed herein, T₂* relaxometry can be effectively employed in the quantification and monitoring of labeled stem cells in cellular therapies. As noted above, T₂* relaxometry is generally performed by multiple gradient echo imaging. However, in tissues containing highly concentrated iron labeled cells, T₂* can be below 2 milliseconds and therefore the signal decay is too rapid for regular gradient echo times. Taking advantage of the relatively long T₂ decay of cell bounded SPIO, the disclosed system/method is employed to measure fast decaying T₂* relaxation using a series of spin echo images. In this illustrative example, the in vivo quantification of short T₂* in rats with iron labeled tumors was investigated.

Sequence Development: Measurement of ultrashort T₂* was achieved by acquiring a series of spin echo images as shown in FIG. 2. The first echo was obtained as a regular spin echo image. The next images were acquired by shifting the readout towards the T₂* decay by steps below 1 millisecond. This allowed sampling of the T₂* decay curve from the spin-echo signal.

In vivo experiment: C8161 melanoma cells were labeled with Feridex-protamine sulfate (FEPro) complexes using procedures labeling procedures as are known in the art. 2×10⁶ FEPro labeled or unlabeled (control) melanoma cells were implanted subcutaneously bilaterally into the flanks of 5 nude rats. MRI was performed approximately two weeks after the inoculation of tumor cells on a 3 T Intera whole-body scanner (Philips Medical System) using a dedicated 7 cm rat solenoid RF-coil. A regular T₂* map was acquired with multiple gradient echo sequence (MGES) [TR/TE=1540/16 ms, 13 echoes, 256×256 matrix, 17 slices, Slice-thickness=1.0 mm, FOV=80 mm, NEX=4]. To measure the short T₂*, five sets of spin echo images were obtained with the readout echo shifted 0 ms, 0.4 ms, 0.8 ms, 1.2 ms and 2.3 ms, respectively, with the following parameters: TR/TE=1000/6.4, 144×144 matrix, 17 slices, Slice-thickness=1.5 mm, FOV=80 mm, NEX=4.

Data analysis: Data analysis was performed using an IDL software tool. T₂ maps were derived using exponential fitting. Both datasets (i.e., regular T₂* map and the short T₂* map) were combined and displayed as T₂* map.

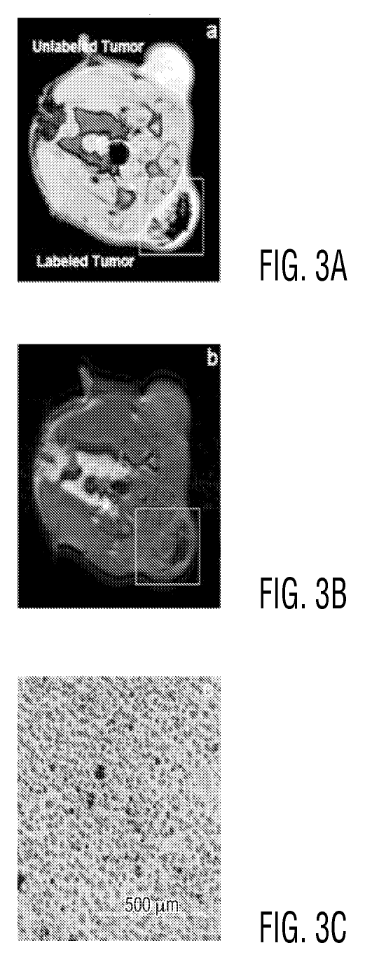

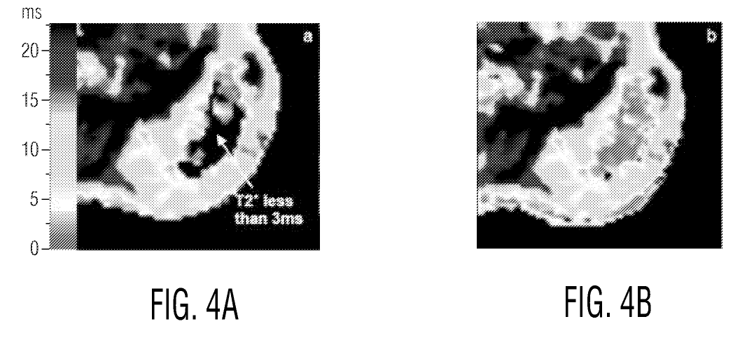

Ultrashort T₂* relaxometry maps and MGES conventional T₂* maps were obtained for 4 rats. FIG. 3a shows an axial gradient echo image of flank tumors in a rat. The signal void in the labeled tumor was induced by highly concentrated iron labeled cells as illustrated in FIG. 3c. However, the spin echo image of the same tumor (FIG. 3b) suffers less signal decay given the relatively long T₂ relaxation time of cell bounded SPIO. The T₂* map measured using MGES (FIG. 4a) illustrates areas of high T₂* values on the tumor border indicative of serial dilution of the FEPro labeling as the tumor grows. The MGES T₂* map failed to detect any signal due to the fast T₂* decay induced by heavily concentrated labeled cells in the center of the tumor. As a comparison, the ultrashort T₂* maps (FIG. 4b) demonstrate T₂* values in the center of the tumor of approximately ≦1 ms, which corresponds to areas of highly concentrated iron labeled cells in FIG. 3a.

Conclusion: This experiment demonstrated the effective measurement of ultrashort T₂* relaxation times in cells and tissues. In vivo MR experiments demonstrate that this method can measure ultrashort T₂* values down to 1 ms or less in highly concentrated iron labeled cells. Combined with the conventional T₂* map, the disclosed technique can be employed to improve the in vivo quantification and monitoring of tissues containing heavily iron labeled cells.

### Example 2

Quantifying the number of labeled stem cells in target tissues in experimental models is of great importance to optimize dose and timing of cellular therapy in clinical trials. SPIO agents are used to label cells to monitor their migration, proliferation and/or homing by MR imaging. R₂*(1/T₂*) relaxation rate is a sensitive parameter for quantitative detection of intracellular SPIO.

In this illustrative example, the quantitative relationship between the number of iron labeled cells and R₂* relaxation rate in a tumor rat model was investigated. More particularly, the quantitative relationship between iron labeled cells and tissue R₂* relaxation rate in a tumor rat model was investigated. The in vivo experiments demonstrated an excellent linear correlation between the number of iron labeled cells and tissue R₂. The data further illustrates that R₂ measurement is a reliable and sensitive approach for the in vivo quantification of iron labeled cells.

C8161 melanoma cells and C6 glioma cells were labeled with Feridex-protamine sulfate (FEPro) complexes using known procedures. Nude rats were implanted subcutaneously bilaterally with 2×10⁶ FEPro labeled and unlabeled (control) melanoma cells (n=4) or 1×10⁶ FEPro labeled and unlabeled C6 glioma cells (n=4). MRI was performed approximately two weeks after the inoculation of the tumor cells on a 3 T Intera whole-body scanner (Philips Medical System) using a dedicated 7 cm rat solenoid RF-coil. A regular R₂* map was acquired with multiple gradient echo sequence [TR/TE=1540/16 ms, 13 echoes, 256×256 matrix, 17 slices, Slice-thickness=1.0 mm, FOV=80 mm, NEX=4]. To measure the R₂* relaxation in tissues with highly concentrated labeled cells, five sets of spin echo images were obtained with the readout echo shifted 0 ms, 0.4 ms, 0.8 ms, 1.2 ms and 2.3 ms respectively [TR/TE=1000/6.4, 144×144 matrix, 17 slices, Slice-thickness=1.5 mm, FOV=80 mm, NEX=4]. R₂* relaxation rates were calculated by exponential fitting using an IDL software tool. Both datasets (i.e., regular R₂* map and R₂* map of tissues with highly concentrated labeled cells) were combined. The R₂* relaxation of the tumor was calculated as the average of pixel-wised R₂* relaxation over the whole tumor volume. The number of labeled cells per mm³ was determined as the number of implanted tumor cells divided by the tumor volume.

Results: Iron labeling did not change the tumor's growth. There was no significant statistical difference in tumor size between labeled and unlabeled tumors. Labeled tumor sizes ranged from 1890 mm³ to 4950 mm³ at the time of imaging, which translates to 325 to 1056 labeled cells per mm³ in eight tumors.

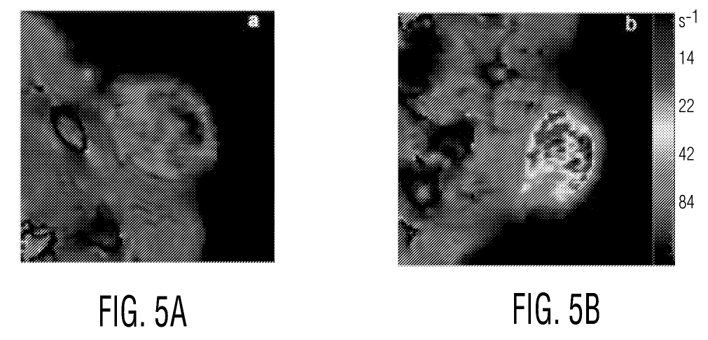

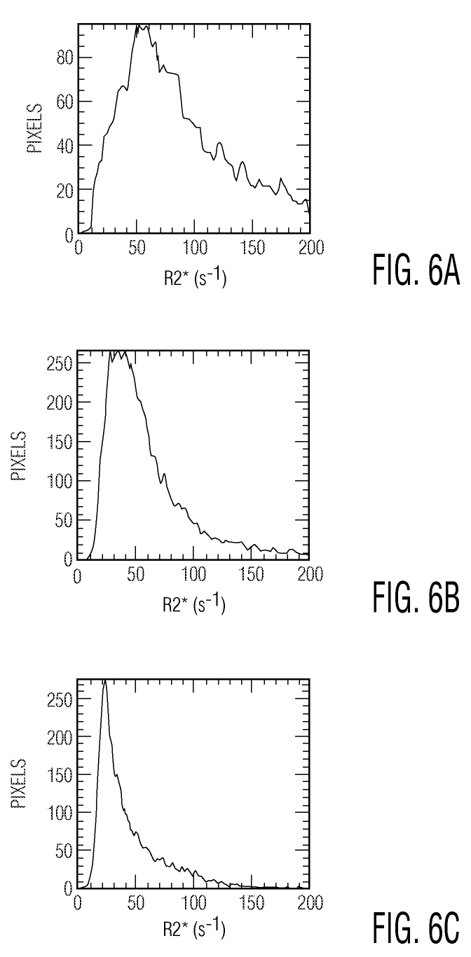

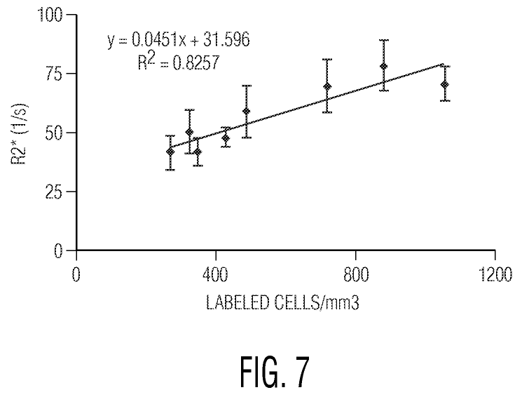

FEPro labeling significantly lengthened the R₂* relaxation rate of the tumor. FIGS. 5a and 5b illustrate R₂* maps from a labeled and an unlabeled tumor, respectively. The effect of iron labeling on R₂* relaxation can be further substantiated by the R* histogram of the tumor with 1056 labeled cells/mm³ (FIG. 6a) and 325 labeled cells/mm³ (FIG. 6b). The labeled tumors developed a much wider R₂ distribution as compared to the control tumor (FIG. 6c). The average R₂* of the tumor demonstrated a very good linear correlation with the number of labeled cells per mm³ (FIG. 7), with a correlation coefficient of 0.91 (p<0.01).

Conclusion: In this illustrative example, the quantitative relationship between the iron labeled cells and tissue R₂* relaxation rate was investigated. Although two different tumor cell lines were used, the in vivo data demonstrated an excellent linear correlation between the number of iron labeled cells and tissue R₂*. The experimental data further illustrated that R₂ measurement is a reliable and sensitive tool for quantification of iron labeled cells. Accordingly, the disclosed systems and methods may be employed for effective quantitative non-invasive assessment of iron labeled cells in vivo.

In sum, the systems and methods of the present disclosure offer significantly enhanced techniques for MR measurement of labeled cells in a variety of applications. Indeed, current investigations in cell trafficking and therapy begin with the injection of large amounts of SPIO labeled cells into a specific site, resulting in very short T₂* in the labeled and surrounding tissues. The disclosed systems and methods facilitate significant improvements in the quantification of labeled cells, despite the ultrashort T₂* decay to be encountered in such tissue systems. The disclosed systems and methods can also be applied to measure ultrashort T₂* of other contrast agents that cause significant difference in T₂ and T₂* relaxation.

Although the present disclosure has been described with reference to exemplary embodiments and implementations thereof, the disclosed systems and methods are not limited to such exemplary embodiments/implementations. Rather, as will be readily apparent to persons skilled in the art from the description provided herein, the disclosed systems and methods are susceptible to modifications, alterations and enhancements without departing from the spirit or scope of the present disclosure. Accordingly, the present disclosure expressly encompasses such modification, alterations and enhancements within the scope hereof.

1. A method for measuring labeled cells, comprising:
labeling cells ex vivo with a contrasting agent;
monitoring migration, proliferation and/or homing of said labeled cells with magnetic resonance (MR) imaging;
measuring T₂* relaxometry having a T₂* decay curve by acquiring a series of spin echo images comprising the steps of:
(a) inducing a first spin echo signal generating a first spin echo image;
(b) inducing multiple spin echo signals generating a series of additional spin echo images from suitable echo shifts towards said T₂* decay; and
(c) deriving T₂* maps using exponential fitting.

2. A method according to claim 1, wherein said contrasting agent is superparamagnetic iron oxide (SPIO).

3. A method according to claim 1, wherein T₂* is ultrashort.

4. A method according to claim 3, wherein T₂* varies from application-to-application, and in certain applications is less than or equal to 2 milliseconds.

5. A method according to claim 1, wherein said first spin echo signal and said second spin echo signal are formed by a first radio frequency (RF) pulse followed by a second RF pulse respectively.

6. A method according to claim 5, wherein said first RF pulse is a 90 degree RF pulse followed by a 180 degree RF pulse.

7. A method according to claim 1, wherein a T₂ decay curve is defined by the relationship: Mₛₛe^{-t/T}².

8. A method according to claim 1, wherein said T₂* decay curve is defined by the relationship: Mₛₛe^{-TE/T2}e^{-(t-TE)/T}²*.

9. A method according to claim 1, wherein said suitable echo shift is done by steps below 1 or 2 milliseconds.

10. A method according to claim 1, wherein said T₂ maps are combined and displayed as an overall T₂ map.

11. A method according to claim 1, wherein the labeled cells are measured in connection with cell trafficking or cell therapy.

12. A system for measuring labeled cells according to claim 1.
